(19) United States
(12) Patent Application Publication
(10) Pub. No.: US 2024/0389591 A1
(43) Pub. Date: Nov. 28, 2024

(54) CRYSTALLINE FORMS OF METHYL 2-[2-[2-BROMO-4-FLUORO-5-[3-METHYL-2,6-DIOXO-4-(TRIFLUOROMETHYL)PYRIMIDIN-1-YL]PHENOXY]PHENOXY]-2-METHOXY-ACETATE

(71) Applicant: BASF SE, Ludwigshafen (DE)

(72) Inventors: Rahul Kaduskar, Navi Mumbai (IN); Martin Viertelhaus, Ludwigshafen (DE); Sunil Kolse, Navi Mumbai (IN); Desislava Slavcheva Petkova, Ludwigshafen (DE); Tobias Seiser, Ludwigshafen (DE); Roland Goetz, Ludwigshafen (DE); Harish Shinde, Navi Mumbai (IN); Henning Urch, Limburgerhof (DE); Ricardo Hugo Pavon Romero, Dos Hermanas (ES)

(21) Appl. No.: 18/685,970

(22) PCT Filed: Aug. 19, 2022
(86) PCT No.: PCT/EP2022/073140

§ 371 Date: Feb. 23, 2024

(30) Foreign Application Priority Data
Aug. 30, 2021 (EP) 21193726.3

(51) Int. Cl. A01N 43/54; A01N 25/04; A01P 13/00; C07D 239/54

(52) U.S. Cl. CPC A01N 43/54; A01N 25/04; A01P 13/00; C07D 239/54

(57) ABSTRACT

The present invention relates to one crystalline form A of methyl 2-[2-[2-bromo-4-fluoro-5-[3-methyl-2,6-dioxo-4-(trifluoromethyl)pyrimidin-1-yl]phenoxy]phenoxy]-2-methoxy-acetate, a process for the production of this crystalline form and formulations for plant protection comprising such form A.

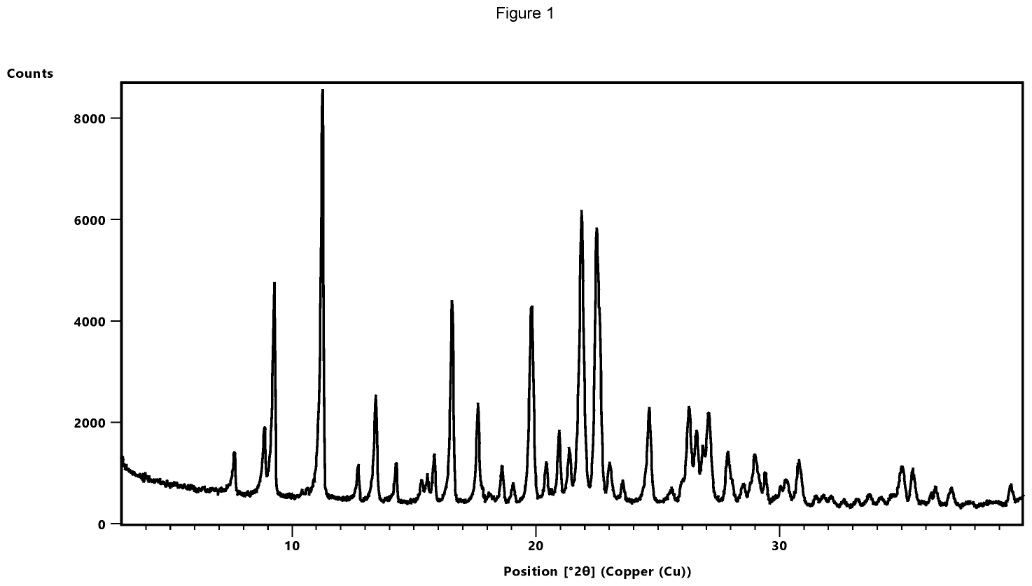

The present invention relates to one crystalline form (hereinafter also referred to as “form A”) of methyl 2-[2-[2-bromo-4-fluoro-5-[3-methyl-2,6-dioxo-4-(trifluoromethyl)pyrimidin-1-yl]phenoxy]phenoxy]-2-methoxy-acetate (hereinafter also referred to as “compound of formula (I)”).

The invention also relates to a process for the production of this crystalline form and formulations (i.e. agrochemical compositions) for plant protection which contain this crystalline form.

The invention also relates to agrochemical compositions comprising at least an auxiliary and at least form A of the compound of formula (I) according to the invention.

The compound of formula (I) is the herbicidal active substance having the chemical formula (I):

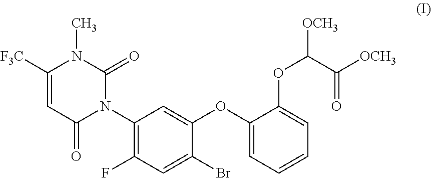 [SMILES: COC(=O)C(OC)Oc1ccccc1Oc1cc(-n2c(=O)cc(C(F)(F)F)n(C)c2=O)c(F)cc1Br]

The compound of formula (I) and a general procedure for its production is known from WO 2021/175689.

WO 2019/101551, WO 2018/019842, EP 1 459 628 and EP 1 470 753 describe structurally similar herbicidal phenyluracils, but do not describe explicitly the compound of formula (I).

For the production of active substances on the industrial scale but also for the formulation of active substances, in many cases knowledge concerning the possible existence of crystalline modifications (also described as crystalline forms) or of solvates of the active substance in question, and knowledge of the specific properties of such modifications and solvates and of methods for their preparation are of decisive importance. A range of active substances can exist in different crystalline but also in amorphous modifications. Polymorphism is the term used in these cases. A polymorph is a solid, crystalline phase of a compound which is characterized by a specific, uniform packing and arrangement of the molecules in the solid.

Different modifications of one and the same active substance can sometimes have different properties, for example differences in the following properties: solubility, vapor pressure, dissolution rate, stability against a phase change into a different modification, stability during grinding, suspension stability, optical and mechanical properties, hygroscopicity, crystal form and size, filterability, density, melting point, stability to decomposition, color, chemical reactivity or biological activity.

The applicant's own attempts to convert the compound of formula (I) into a crystalline solid by crystallization at first resulted in amorphous products, which could only be handled with difficulty.

It has now surprisingly been found that by suitable processes a previously unknown crystalline, stable modification of the compound of formula (I), which do not display the disadvantages of the amorphous compound of formula (I), is obtained in high purity. This modification is described below.

In addition, the crystal form A according to the invention is easier to handle than the previously known amorphous compound of formula (I), since during production it is obtained in the form of discrete crystals or crystallites.

The stability of formulations which contain the compound of formula (I) in form A is also markedly higher than the stability of formulations which contain the compound of formula (I) in amorphous form.

The term “pure form A” should be understood to mean that the proportion of the modification in question, based on the total quantity of the compound of formula (I), is at least 80 wt. %, preferably at least 90 wt. % and in particular at least 95 wt. %.

The compound of formula (I) can be prepared by standard processes of organic chemistry, for example by reaction of compounds of formula (II) with alkylating agents of formula (III) in the presence of a base in analogy to known processes (e.g. WO 11/137088):

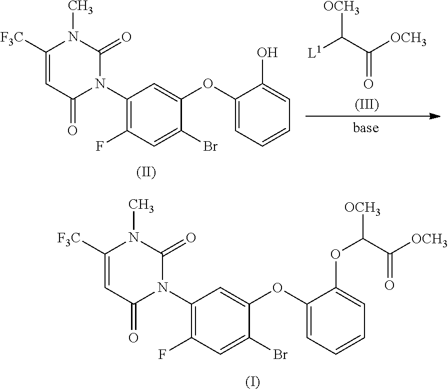

Within the alkylating agents of formula (III), L¹ is a leaving group such as halogen.

The alkylating agents of formula (III) are commercially available or can be prepared by known methods (e.g. WO 11/137088).

Compounds of formula (II) can be prepared by deprotection of the respective compounds of formula (VI):

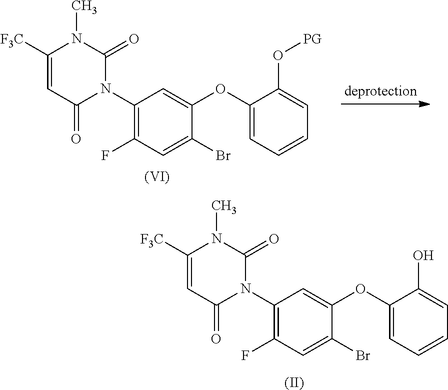

Within the compounds of formula (VI) “PG” is a protecting group selected from the group consisting of C₁-C₆-alkyl or (tri-C₁-C₆-alkyl)silyl-C₁-C₄-alkyl.

For example, the compounds of formula (II) can be prepared by treating the compounds of formula (VI), wherein “PG” is methyl, with boron tribromide in a solvent such as dichloromethane at temperatures ranging from 0° C. to 150° C.

Compounds of formula (VI) can be prepared by alkylation of the corresponding NH-uracil. Such alkylation can be conducted in analogy to known processes (e.g. WO 05/054208; WO 06/125746).

As alkylation reagents commercially available C₁-C₆-alkylhalides and alkinylhalides can be used.

The corresponding NH-uracil can be prepared by reaction of amines of formula (VII) with an oxazinone of formula (VIII):

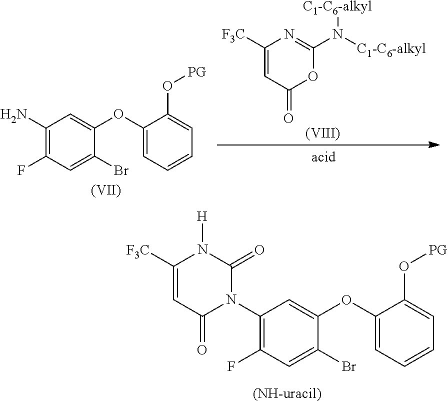

Within the amines of formula (VII) the group “PG” is a protecting group as defined above for the compounds of formula (VI).

The reaction may in principle be carried out in substance. However, preference is given to reacting the amines of formula (VII) with the oxazinones of formula (VIII) in an organic solvent. Suitable in principle are all solvents which are capable of dissolving the amines of formula (VII) and the oxazinones of formula (VIII) at least partly, and preferably fully under reaction conditions.

Examples of suitable solvents are halogenated hydrocarbons such as dichloromethane, 1,2-dichloroethane, chloroform, carbon tetrachloride and chlorobenzene, ethers such as diethyl ether, diisopropyl ether, tert.-butyl methylether (TBME), dioxane, anisole and tetrahydrofuran (THF), esters such as ethyl acetate and butyl acetate; nitriles such as acetonitrile and propionitrile, ketones such as acetone, methyl ethyl ketone, diethyl ketone, tert-butyl methyl ketone, cyclohexanone; organic acids like formic acid, acetic acid, propionic acid, oxalic acid, methylbenzenesulfonic acid, benzenesulfonic acid, camphorsulfonic acid, citric acid, trifluoroacetic acid as well as dipolar aprotic solvents such as sulfolane, dimethylsulfoxide, N,N-dimethylformamide (DMF), N,N-dimethylacetamide (DMAC), 1,3-dimethyl-2-imidazolidinone (DMI), N,N′-dimethylpropylene urea (DMPU), dimethyl sulfoxide (DMSO) and 1-methyl-2 pyrrolidinone (NMP).

It is also possible to use mixtures of the solvents mentioned.

As acids anorganic acids like hydrochloric acid, hydrobromic acid or sulfuric acid, as well as organic acids like formic acid, acetic acid, propionic acid, oxalic acid, methylbenzenesulfonic acid, benzenesulfonic acid, camphorsulfonic acid, citric acid, trifluoroacetic acid, can be used.

The acids are generally employed in equimolar amounts, in excess or, if appropriate, be used as solvent, however they can also be employed in catalytic amounts.

The oxazinones of formula (VIII) required for the preparation of compounds of formula (VI) are commercially available or can be prepared by known methods.

The amines of formula (VII) required for the preparation of compounds of formula (VI) or their respective NH-uracils can be prepared from the compounds of formula (IX):

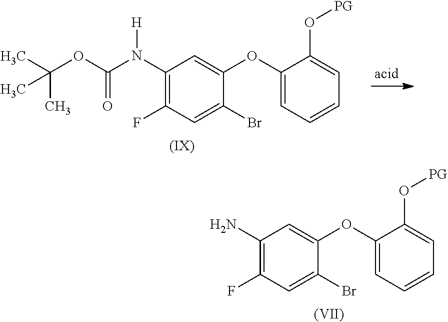

Within the compounds of formula (IX) the group “PG” is a protecting group as defined above for the compounds of formula (VI).

Examples of suitable solvents are halogenated hydrocarbons such as dichloromethane, 1,2-dichloroethane, chloroform, carbon tetrachloride and chlorobenzene, ethers such as diethyl ether, diisopropyl ether, tert.-butyl methylether (TBME), dioxane, anisole and tetrahydrofuran (THF); nitriles such as acetonitrile and propionitrile, ketones such as acetone, methyl ethyl ketone, diethyl ketone, tert-butyl methyl ketone, cyclohexanone; alcohols such as methanol, ethanol, n-propanol, isopropanol, n-butanol and tert.-butanol, organic acids like formic acid, acetic acid, propionic acid, oxalic acid, methylbenzenesulfonic acid, benzenesulfonic acid, camphorsulfonic acid, citric acid, trifluoroacetic acid as well as dipolar aprotic solvents such as sulfolane, dimethylsulfoxide, N,N-dimethylformamide (DMF), N,N-dimethylacetamide (DMAC), 1,3-dimethyl-2-imidazolidinone (DMI), N,N′-dimethylpropylene urea (DMPU), dimethyl sulfoxide (DMSO) and 1-methyl-2 pyrrolidinone (NMP).

As acids inorganic acids like hydrochloric acid, hydrobromic acid or sulfuric acid, as well as organic acids like formic acid, acetic acid, propionic acid, oxalic acid, methylbenzenesulfonic acid, benzenesulfonic acid, camphorsulfonic acid, citric acid, trifluoroacetic acid, can be used.

The acids are generally employed in equimolar amounts, in excess or, if appropriate, be used as solvent, however they can also be employed in catalytic amounts.

The compounds of formula (IX) required for the preparation of compounds of formula (VII) can be prepared by reduction followed by a Sandmeyer reaction from the corresponding nitro compounds of formula (X):

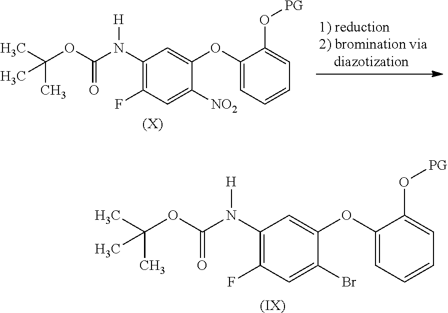

Within the nitro compounds of formula (X) the group “PG” is a protecting group as defined above for the compounds of formula (VI).

Reduction of the nitro group of nitro compound of formula (X) can be carried out by catalytic hydrogenation in hydrogen gas at a pressure of 70 to 700 kPa, preferably 270 to 350 kPa, in the presence of a metal catalyst such as palladium supported on an inert carrier such as activated carbon, in a weight ratio of 5 to 20% of metal to carrier, suspended in a solvent such as ethanol at ambient temperature.

Bromination of the resulting amine is facilitated by diazotization with an alkyl nitrite (e.g. iso-amyl nitrite) followed by treatment with a copper (I) bromide and/or copper (II) bromide in a solvent such as acetonitrile at a temperature ranging from 0° C. to the reflux temperature of the solvent to give the corresponding compound of formula (IX).

The nitro compounds of formula (X) required for the preparation of compounds of formula (IX) can be prepared by reaction of compounds of formula (XI) with compounds of formula (XII) in the presence of a base:

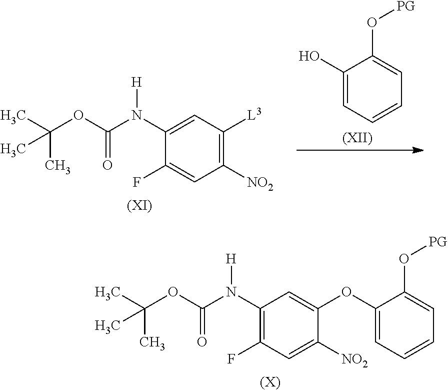

Within the compounds of formula (XI) L³ is a leaving group such as halogen.

The reaction is carried out in an organic solvent.

Examples of suitable solvents are halogenated hydrocarbons such as dichloromethane, 1,2-dichloroethane, chloroform, carbon tetrachloride and chlorobenzene, ethers such as diethyl ether, diisopropyl ether, tert.-butyl methylether (TBME), dioxane, anisole and tetrahydrofuran (THF), nitriles such as acetonitrile and propionitrile, as well as dipolar aprotic solvents such as sulfolane, dimethylsulfoxide, N,N-dimethylformamide (DMF), N,N-dimethylacetamide (DMAC), 1,3-dimethyl-2-imidazolidinone (DMI), N,N′-dimethylpropylene urea (DMPU), dimethyl sulfoxide (DMSO) and 1-methyl-2 pyrrolidinone (NMP).

It is also possible to use mixtures of the solvents mentioned.

Examples of suitable bases include metal-containing bases and nitrogen-containing bases. Examples of suitable metal-containing bases are inorganic compounds such as alkali metal and alkaline earth metal hydroxides, and other metal hydroxides, such as lithium hydroxide, sodium hydroxide, potassium hydroxide, magnesium hydroxide, calcium hydroxide and aluminum hydroxide; alkali metal and alkaline earth metal oxide, and other metal oxides, such as lithium oxide, sodium oxide, potassium oxide, magnesium oxide, calcium oxide and magnesium oxide, iron oxide, silver oxide; alkali metal and alkaline earth metal hydrides such as lithium hydride, sodium hydride, potassium hydride and calcium hydride, alkali metal amides such as lithium amide, sodium amide and potassium amide, alkali metal and alkaline earth metal carbonates such as lithium carbonate, sodium carbonate, potassium carbonate, magnesium carbonate, and calcium carbonate, as well as alkali metal hydrogen carbonates (bicarbonates) such as lithium hydrogen carbonate, sodium hydrogen carbonate, potassium hydrogen carbonate; alkali metal and alkaline earth metal phosphates such as potassium phosphate, calcium phosphate; metal organic compounds, preferably alkali metal alkyls such as methyl lithium, butyl lithium and phenyl lithium, alkyl magnesium halides such as methyl magnesium chloride as well as alkali metal and alkaline earth metal alkoxides such as potassium tert-butoxide, potassium tert-pentoxide; and furthermore organic bases, such as tertiary amines such as trimethylamine, triethylamine, diisopropylethylamine and N-methylpiperidine, pyridine, substituted pyridines such as collidinge, lutidine, N-methylmorpholine and 4-dimethylaminopyridine and also bicyclic amines.

The bases are generally employed in equimolar amounts or in excess; however they can also be employed as solvent, or, if appropriate, in catalytic amounts.

The compounds of formula (XII) required for the preparation of nitro compounds of formula (X) are commercially available or can be prepared by known methods.

The compounds of formula (XI) required for the preparation of nitro compounds of formula (X) can be prepared from compounds of formula (XIII):

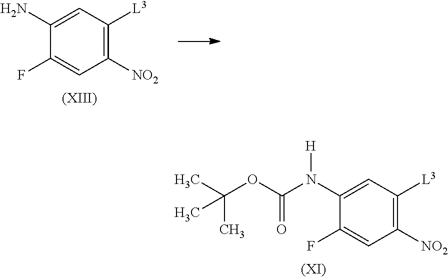

The reaction can be carried out by adding bis(1,1-dimethylethyl) dicarbonate (CAS 24424-99-5) to compounds of formula (XIII) in an organic solvent. The addition of a base can be advantages.

Examples of suitable solvents are halogenated hydrocarbons such as dichloromethane, 1,2-dichloroethane, chloroform.

Examples of suitable bases are nitrogen-containing bases such as 4-(dimethylamino)pyridine (DMAP), 1,8-diazabicyclo[5.4.0]undec-7-ene (DBU) or 1,5-diazabicyclo[4.3.0]non-5-ene (DBN).

The compounds of formula (XIII) required for the preparation of compounds of formula (XI) are commercially available or can be prepared by known methods.

Accordingly, the present invention relates to the crystalline form (A) of the compound of formula (I). Also an object is a compound of formula (I) which at least 80 wt %, preferably at least 90 wt. %, in particular at least 95%, consists of the crystalline form (A).

The form (A) according to the invention can be identified by X-ray powder diffractometry based on its diffraction diagram.

An X-ray powder diffraction diagram recorded using Cu—Kα radiation (1.54178 Å) at 25° C. shows at least 3, preferably at least 5, in particular at least 7, and especially all the reflections quoted in the following table 1 as 26 values (or as interplanar spacings d):

TABLE 1

| 2θ | d [Å] |
|---|---|
| 7.6 ± 0.2 | 11.6 ± 0.3 |
| 8.9 ± 0.2 | 10.0 ± 0.3 |
| 9.3 ± 0.2 | 9.5 ± 0.3 |
| 11.2 ± 0.2 | 7.9 ± 0.2 |
| 12.7 ± 0.2 | 7.0 ± 0.2 |
| 13.4 ± 0.2 | 6.59 ± 0.1 |
| 14.3 ± 0.2 | 6.21 ± 0.1 |
| 15.8 ± 0.2 | 5.60 ± 0.08 |
| 16.6 ± 0.2 | 5.35 ± 0.07 |
| 17.6 ± 0.2 | 5.03 ± 0.06 |
| 18.6 ± 0.2 | 4.77 ± 0.06 |
| 19.1 ± 0.2 | 4.66 ± 0.06 |
| 19.8 ± 0.2 | 4.48 ± 0.05 |
| 20.4 ± 0.2 | 4.35 ± 0.05 |
| 21.0 ± 0.2 | 4.24 ± 0.05 |
| 21.4 ± 0.2 | 4.16 ± 0.05 |
| 21.9 ± 0.2 | 4.06 ± 0.04 |
| 22.5 ± 0.2 | 3.95 ± 0.04 |
| 22.6 ± 0.2 | 3.93 ± 0.04 |
| 23.0 ± 0.2 | 3.86 ± 0.04 |
| 23.6 ± 0.2 | 3.78 ± 0.04 |
| 24.7 ± 0.2 | 3.61 ± 0.04 |
| 25.5 ± 0.2 | 3.49 ± 0.04 |
| 26.3 ± 0.2 | 3.39 ± 0.03 |
| 26.6 ± 0.2 | 3.35 ± 0.03 |
| 27.1 ± 0.2 | 3.29 ± 0.03 |
| 27.9 ± 0.2 | 3.20 ± 0.03 |
| 28.5 ± 0.2 | 3.13 ± 0.03 |
| 29.0 ± 0.2 | 3.08 ± 0.03 |
| 29.4 ± 0.2 | 3.04 ± 0.03 |

Preferably, an X-ray powder diffraction diagram recorded using Cu—Kα radiation (1.54178 Å) at 25° C. shows the following reflection(s) as 26 values: 9.3, 11.2, 13.4, 16.6, 19.8, 21.9 and 22.5°2θ (all values with ±0.2°2θ);
particularly preferred 9.3, 11.2, 13.4, 16.6, 19.8, 21.9, 22.5 and 26.3°2θ (all values with ±0.2°2θ); and
especially preferred 7.6, 8.9, 9.3, 11.2, 12.7, 13.4, 14.3, 15.8, 16.6, 17.6, 19.8, 21.9, 22.5, 23.0 and 26.3°2θ (all values with ±0.2°2θ).

The preparation of the compound of formula (I) used for the production of the form A can be effected as described above.

The production of the form (A) of the compound of formula (I) according to the invention can be effected by
crystallization from a solution of the compound of formula (I)
crystallization from a solution of the compound of formula (I) by adding seed crystals
crystallization from a solution of the compound of formula (I) in a suitable organic solvent
crystallization from a solution of the compound of formula (I) by cooling
crystallization from a solution of the compound of formula (I) by evaporation
a combination of the above-mentioned methods.

The solution of compound of formula (I) can for example be prepared by the following methods:
(1) Dissolution of the compound of formula (I), preferably in a form different from form (A), in one of the solvents mentioned below, or
(2) Preparation of the compound of formula (I) by a chemical reaction and transfer of the reaction mixture, if necessary after removal of reagents and/or side products, into an organic solvent suitable according to the invention.

For the preparation of the solution by dissolution of the compound of formula (I), essentially any known form of compound of formula (I) can be used. Often amorphous compound of formula (I) or a mixture of amorphous and crystalline compound of formula (I) will be used.

The dissolution of the compound of formula (I) is usually effected at temperatures in the range from 20 to 100° C. In one embodiment of the invention, the dissolution of the compound of formula (I) is effected at elevated temperature, in particular at 30 to 80° C., and naturally the temperature used for dissolution will not exceed the boiling point of the solvent.

The crystallization is often effected at temperatures in the range from 20° C. to 50° C. It is, however, preferred to effect crystallisation at temperatures of at most 45° C., in particular at most 30° C. and more preferably at most 25° C.

The solution of the compound of formula (I) can also be prepared by transferring a reaction mixture obtained by a chemical reaction, which contains the compound of formula (I), if necessary after removal of reagents and/or side products, into an organic solvent suitable according to the invention. This can be effected in such a manner that the reaction is performed in an organic solvent or solvent mixture which consists at least partly, preferably at least 30 wt. %, of a solvent suitable for the crystallization and, if necessary a workup is performed during which excess reagents and any catalysts present and any unsuitable solvents present, for example water and/or methanol, are removed. The preparation of a solution of the compound of formula (I) by chemical reaction of a suitable precursor of compound of formula (I) can be effected by analogy to the methods which are described in the state of the art cited at the beginning, to which full reference is hereby made.

The production of the form (A) of the compound of formula (I) according to the invention is effected by crystallization from a solution of the compound of formula (I) in a suitable organic solvent.

Suitable solvents for the crystallization of form (A) are aliphatic hydrocarbons such as pentane, hexane, heptane, cyclohexane, nitromethane and mixtures of C₅-C₃-alkanes, aromatic hydrocarbons such as benzene, toluene, cresols, o-, m- and p-xylene, halogenated hydrocarbons such as dichloromethane, 1,2-dichloroethane, chloroform, carbon tetrachloride and chlorobenzene, ethers such as diethyl ether, diisopropyl ether, tert.-butyl methylether (TBME), dioxane, anisole and tetrahydrofuran (THF), esters such as ethyl acetate and butyl acetate; nitriles such as acetonitrile and propionitrile, ketones such as acetone, methyl ethyl ketone, diethyl ketone, tert-butyl methyl ketone, cyclohexanone; alcohols such as methanol, ethanol, n-propanol, isopropanol, n-butanol and tert.-butanol, dipolar aprotic solvents such as sulfolane, dimethylsulfoxide, N,N-dimethylformamide (DMF), N,N-dimethylacetamide (DMAC), 1,3-dimethyl-2-imidazolidinone (DMI), N,N′-dimethylpropylene urea (DMPU), dimethyl sulfoxide (DMSO) and 1-methyl-2 pyrrolidinone (NMP), water, and mixtures of the afore-mentioned solvents.

Preferred solvents are aliphatic hydrocarbons such as heptane, and alcohols such as methanol, isopropanol and tert.-butanol, water and mixtures of these.

In order to obtain form (A) of the compound of formula (I), the crystallization is preferably effected at most 45° C., in particular from −15° C. to 30° C.

Crystallization of form (A) is preferably effected under controlled conditions, i.e. the conditions of the crystallization are chosen to achieve a slow crystallization rate.

For this, in a first step i) a solution of the compound of formula (I) in one of the aforesaid organic solvents is prepared, and then in a second step ii) crystallization of the compound of formula (I) is effected.

The concentration of compound of formula (I) in the solution used for the crystallization naturally depends on the nature of the solvent and the solution temperature and often lies in the range from 5 to 3000 g/l. Suitable conditions can be determined by the person skilled in the art by routine experiments.

Preferably the solution used for the crystallization contains compound of formula (I) in a purity of at least 50%, often at least 75%, in particular at least 90%, i.e. the content of organic impurities which are not organic solvents is not more than 50 wt. %, often not more than 25 wt. %, and in particular not more than 10 wt. %, based on the compound of formula (I) present dissolved in the solvent.

The solution used for the crystallization is preferably essentially free from solvents other than those stated. In this context, “essentially free” means that the concentration of other solvents in the compound of formula (I)-containing solution does not exceed 10 wt. %, often 5 wt. %, based on the total quantity of solvent.

The crystallization of form (A) of compound of formula (I) can be effected as follows, for example
by cooling of the solution which contains the dissolved compound of formula (I),
by allow the solution, which contains the dissolved compound of formula (I), to stand for some time at room temperature,
by addition of a solubility-decreasing solvent to the solution which contains the dissolved compound of formula (I), in particular by addition of a nonpolar organic solvent or by addition of water,
by concentration of the solution which contains the dissolved compound of formula (I),
by stirring of a suspension of compound of formula (I) and preferably seeding with the form (A) of compound of formula (I), or
by a combination of the aforesaid measures.

The crystallization is as a rule carried out until at least 50 wt. %, preferably at least 85 wt. %, of the compound of formula (I) used crystallizes out.

If the crystallization of form (A) is effected by cooling, the cooling rate is preferably less than 10 K/min.

The crystallization of form (A) can be promoted or accelerated by seeding with seed crystals of form (A), for example by adding seed crystals of form (A) before or during the crystallization.

If seed crystals are added during the crystallization, the quantity thereof is typically 0.001 to 10 wt. %, often 0.005 to 5 wt. %, in particular 0.01 to 1 wt. % and especially 0.02 to 0.2 wt. %, based on the dissolved compound of formula (I).

If the crystallization is performed in the presence of seed crystals of form (A), these are preferably only added at a temperature at which the saturation concentration of the compound of formula (I) in the solvent in question has been reached, i.e. at or below that temperature at which the dissolved quantity of compound of formula (I) forms a saturated solution in the solvent in question. The person skilled in the art can determine the temperature dependence of the saturation concentration in a solvent in routine experiments.

Alternatively, the crystallization can also be effected by addition of a “non-solvent” (i.e. a solubility decreasing solvent) e.g. by addition of a nonpolar solvent or by addition of water, for example from 5 to 95 vol. %, in particular 20 to 80 vol. % and especially from 30 to 60 vol. %, based on the volume of the polar organic solvent or solvent mixture used for dissolution of the compound of formula (I). The addition of the nonpolar solvent or the addition of water are preferably effected over a prolonged period, for example over a period from 5 mins to 3 hrs, in particular over a period from 10 mins to 2 hrs. If the crystallization of form (A) is effected by the addition of a “non-solvent”, the addition of the non-solvent is preferably at a slow rate, e.g. less than 10% v/v per minute, based on the volume of the compound of formula (I) solution. Often the addition will be done in such a manner that the nonpolar solvent or water is added until the discernable onset of the crystallization and the mixture thus obtained is then left for a time, during which the crystallization of the form (A) proceeds. If necessary, the mixture can then be cooled for completion of the crystallization.

In particular, the addition of the nonpolar solvent or the addition of water and the addition of seed crystals can be combined.

The addition of the nonpolar solvent can be effected in the form of a pure nonpolar solvent or in the form of a mixture of a nonpolar solvent with a solvent used for the dissolution. Examples of nonpolar solvents are aliphatic and cycloaliphatic hydrocarbons with preferably 5 to 10 C atoms such as pentane, hexane, cyclopentane, cyclohexane, isohexane, heptane, cycloheptane, octane, decane or mixtures thereof.

The isolation of the form (A) from the crystallization product, i.e. the separation of the form (A) from the mother liquor, is effected by usual techniques for the separation of solid components from liquids, for example by filtration, centrifugation or by decantation. As a rule, the isolated solid will be washed, for example with the solvent used for the crystallization, with water or with a mixture of the organic solvent used for the crystallization with water. The washing can be effected in one or more steps, washing with water often being used in the last washing step. The washing is typically effected at temperatures below 30° C., often below 25° C. and in particular below 20° C., in order to keep the loss of valuable product as small as possible. Next, the form (A) obtained can be dried and then supplied for further processing. Often, however, the moist active substance obtained after washing, in particular an active substance moist with water, will be supplied directly for the further processing.

By means of the crystallization according to the invention, the form (A) is obtained with a compound of formula (I) content of as a rule at least 90 wt. %, often 94 wt. %, in particular at least 96 wt. %.

The content of form (A), based on the total quantity of compound of formula (I), is typically at least 90% and often at least 95% or at least 96%.

### EXAMPLE 1

Preparation of Form a of Compound of Formula (I) by Crystallization from an Organic Solvent, Addition of a 2^{nd} Solvent and Cooling:

A suspension of amorphous solid (10.5 g) of compound of formula (I) in methanol (4.5 g) was heated to 50° C. The solution was cooled to 25° C. in 1 hour and subsequently cooled to −10° C. in 3 hours. A sticky solid was obtained. After addition of heptane (20 ml) the mixture was warmed to 0° C.; the supernatant layer was decanted, and another portion of heptane (20 ml) was added. The gummy mass was scratched with a spatula while warming it to 20° C. Again, the supernatant layer was decanted, and another portion of heptane (20 ml) was added. The mixture was cooled to 0° C. and stirred for 5 min, the supernatant layer was decanted. Formation of a solid was observed. Methanol (20 ml) was added, and the mixture stirred for 1 h at RT (150 RPM). The slurry was filtered and dried under vacuum to obtain 1 g of white crystalline material.

Based on the characteristic reflections, form A was identified.

Peak positions observed: 7.6, 8.9, 9.3, 11.2, 12.7, 13.5, 14.3, 15.4, 15.8, 16.6, 17.6, 18.7, 19.0, 19.8, 20.4, 20.9, 21.3, 21.8, 22.6, 24.5, 26.2, 27.8, 28.9 and 29.4°2θ (all values with ±0.2°2θ).

### EXAMPLE 2

Preparation of Form a of Compound of Formula (I) by Crystallization from a Mixture of an Organic Solvent with Water with Evaporation Crystallization:

After addition of methanol (17.5 g) and water (3.5 g) to the amorphous solid (15 g) the formation of white lumps was observed. The mixture was heated to 60° C. for 15 minutes and cooled down to room temperature in 2 hours. A well to stir suspension was obtained which was stirred at ambient temperature for 2 hours. The solid obtained was filtered and dried under vacuum to obtain 13 g of white crystalline material.

Based on the characteristic reflections, form A was identified.

Peak positions observed: 7.6, 8.9, 9.3, 11.2, 12.7, 13.4, 14.3, 15.8, 16.6, 17.6, 18.6, 19.8, 20.4, 21.0, 21.3, 21.8, 22.5, 23.0, 24.6, 26.2, 27.1, 27.9, 29.0, 29.4, 30.8, 35.0 and 35.4°2θ (all values with ±0.2°2θ).

### EXAMPLE 3

Preparation of Form a of Compound of Formula (I) by Crystallization from an Organic Solvent and Addition of Seed Crystals:

A suspension of the compound of formula (I) as amorphous solid (15 g) in methanol (17.5 g) was stirred for 15 minutes at 50° C. and then cooled over 1 hour to 25° C. To the obtained clear solution were added seeding crystals from example 1 and the suspension was stirred for 16 hours at 25° C. To the obtained solid methanol was added (10 g) and the mixtures was stirred at 25° C. for 2 hours. Another portion of methanol (25 g) was added, the mixture was stirred for 10 minutes and filtered. The obtained solid was dried under vacuum to give a white solid (13.3 g).

Based on the characteristic reflections, form A was identified.

Peak positions observed: 7.6, 8.9, 9.3, 11.2, 12.7, 13.4, 14.3, 15.8, 16.6, 17.6, 18.6, 19.1, 19.8, 20.4, 21.0, 21.4, 21.9, 22.5, 23.0, 23.6, 24.7, 25.5, 26.3, 26.6, 27.1, 27.9, 28.5, 29.0, 29.4, 30.3, 30.8 and 33.7°2θ (all values with ±0.2°2θ).

Just like the known amorphous compound of formula (I), the form A of compound of formula (I) are suitable as herbicides, however it is superior to this as regards its handling and formulation properties, as well as its herbicidal activity

The invention thus also relates to plant protection agents containing the crystalline form A and additives usual for the formulation of plant protection agents, in particular plant protection agents in the form of aqueous suspension concentrates (so-called SC's) or non-aqueous suspension concentrates (so-called OD's), and plant protection agents in the form of powders (so-called WP's) and granules (so-called WG's) dispersible in water.

The invention also relates to a process for combating undesired plant growth, which is characterized in that the form A of compound of formula (I), preferably as a suitable active substance preparation, is used on plants, their habitat and/or on seeds.

The compounds of the formula (I) in its form A and the plant protection agents which contain the compound of formula (I) in the form A have an outstanding herbicidal activity against undesired vegetation, i.e. against a broad spectrum of economically important harmful monocotyledonous and dicotyledonous weeds.

Mentioned below are some representatives of monocotyledonous and dicotyledonous weeds, which can be controlled by form A of the compound of formula (I), or the formulations comprising form A of the compound of formula (I), without the enumeration being a restriction to certain species.

Preferably form A of the compound of formula (I), or the formulations comprising form A of the compound of formula (I) are used to control monocotyledonous weeds.

Examples of monocotyledonous weeds on which form A of the compound of formula (I), or the formulations comprising form A of formula (I) act efficiently are selected from the genera Hordeum spp., Echinochloa spp., Poa spp., Bromus spp., Digitaria spp., Eriochloa spp., Setaria spp., Pennisetum spp., Eleusine spp., Eragrostis spp., Panicum spp., Lolium spp., Brachiaria spp., Leptochloa spp., Avena spp., Cyperus spp., Axonopris spp., Sorghum spp., and Melinus spp.

Preferred examples of monocotyledonous weeds on which form A of formula (I), or the formulations comprising form A of formula (I) act efficiently are selected from the species Hordeum murinum, Echinochloa crus-galli, Poa annua, Bromus rubens L., Bromus rigidus, Bromus secalinus L., Digitaria sanguinalis, Digitaria insularis, Eriochloa gracilis, Setaria faberi, Setaria viridis, Pennisetum glaucum, Eleusine indica, Eragrostis pectinacea, Panicum miliaceum, Lolium multiflorum, Brachiaria platyphylla, Leptochloa fusca, Avena fatua, Cyperus compressus, Cyperus esculentes, Axonopris offinis, Sorghum halapense, and Melinus repens.

Especially preferred examples of monocotyledonous weeds on which form A of formula (I), or the formulations comprising form A of formula (I) act efficiently are selected from the species Echinochloa spp., Digitaria spp., Setaria spp., Eleusine spp. and Brachiarium spp.

Also preferably form A of formula (I), or the formulations comprising form A of formula (I) are used to control dicotyledonous weeds.

Examples of dicotyledonous weeds on which form A of formula (I), or the formulations comprising form A of formula (I) act efficiently are selected from the genera Amaranthus spp., Erigeron spp., Conyza spp., Polygonum spp., Medicago spp., Mollugo spp., Cyclospermum spp., Stellaria spp., Gnaphalium spp., Taraxacum spp., Oenothera spp., Amsinckia spp., Erodium spp., Erigeron spp., Senecio spp., Lamium spp., Kochia spp., Chenopodium spp., Lactuca spp., Malva spp., Ipomoea spp., Brassica spp., Sinapis spp., Urtica spp., Sida spp, Portulaca spp., Richardia spp., Ambrosia spp., Calandrinia spp., Sisymbrium spp., Sesbania spp., Capsella spp., Sonchus spp., Euphorbia spp., Helianthus spp., Coronopus spp., Salsola spp., Abutilon spp., Vicia spp., Epilobium spp., Cardamine spp., Picris spp., Trifolium spp., Galinsoga spp., Epimedium spp., Marchantia spp., Solanum spp., Oxalis spp., Metricaria spp., Plantago spp., Tribulus spp., Cenchrus spp. Bidens spp., Veronica spp., and Hypochaeris spp.,

Preferred examples of dicotyledonous weeds on which form A of formula (I), or the formulations comprising form A of formula (I) act efficiently are selected from the species Amaranthus spinosus, Polygonum convolvulus, Medicago polymorpha, Mollugo verticillata, Cyclospermum leptophyllum, Stellaria media, Gnaphalium purpureum, Taraxacum officinale, Oenothera laciniata, Amsinckia intermedia, Erodium cicutarium, Erodium moschatum, Erigeron bonariensis (Conyza bonariensis), Senecio vulgaris, Lamium amplexicaule, Erigeron canadensis, Polygonum aviculare, Kochia scoparia, Chenopodium album, Lactuca serriola, Malva parviflora, Malva neglecta, Ipomoea hederacea, Ipomoea lacunose, Brassica nigra, Sinapis arvensis, Urtica dioica, Amaranthus blitoides, Amaranthus retroflexus, Amaranthus hybridus, Amaranthus lividus, Sida spinosa, Portulaca oleracea, Richardia scabra, Ambrosia artemisiifolia, Calandrinia caulescens, Sisymbrium irio, Sesbania exaltata, Capsella bursa-pastoris, Sonchus oleraceus, Euphorbia maculate, Helianthus annuus, Coronopus didymus, Salsola tragus, Abutilon theophrasti, Vicia benghalensis L., Epilobium paniculatum, Cardamine spp, Picris echioides, Trifolium spp., Galinsoga spp., Epimedium spp., Marchantia spp., Solanum spp., Oxalis spp., Metricaria matriccarioides, Plantago spp., Tribulus terrestris, Salsola kali, Cenchrus spp., Bidens bipinnata, Veronica spp., and Hypochaeris radicata.

Especially preferred examples of dicotyledonous weeds on which form A of formula (I), or the formulations comprising form A of formula (I) act efficiently are selected from the species Amaranthus spp., Erigeron spp., Conyza spp., Kochia spp. and Abutilon spp.

Depending on the application method in question, form A of the compound of formula (I) or the agrochemical compositions containing form A can also be used in a further number of crop plants for the elimination of undesired vegetation.

According to the invention all the crop plants (cultivated plants) mentioned herein are understood to comprise all species, subspecies, variants and/or hybrids which belong to the respective cultivated plants, including but not limited to winter and spring varieties, in particular in cereals such as wheat and barley, as well as oilseed rape, e.g. winter wheat, spring wheat, winter barley etc.

For example, corn is also known as Indian corn or maize (Zea mays) which comprises all kinds of corn such as field corn and sweet corn. According to the invention all maize or corn subspecies and/or varieties are comprised, in particular flour corn (Zea mays var. amylacea), popcorn (Zea mays var. everta), dent corn (Zea mays var. indentata), flint corn (Zea mays var. indurata), sweet corn (Zea mays var. saccharata and var. rugosa), waxy corn (Zea mays var. ceratina), amylomaize (high amylose Zea mays varieties), pod corn or wild maize (Zea mays var. tunicata) and striped maize (Zea mays var. japonica).

Further, most soybean cultivars are classifiable into indeterminate and determinate growth habit, whereas Glycine soja, the wild progenitor of soybean, is indeterminate (PNAS 2010, 107 (19) 8563-856). The indeterminate growth habit (Maturity Group, MG 00 to MG 4.9) is characterized by a continuation of vegetative growth after flowering begins whereas determinate soybean varieties (Maturity Group, (MG) 5 to MG 8) characteristically have finished most of their vegetative growth when flowering begins. According to the invention all soybean cultivars or varieties are comprised, in particular indeterminate and determinate cultivars or varieties.

Examples of suitable crops are the following: Allium cepa, Ananas comosus, Arachis hypogaea, Asparagus officinalis, Avena sativa, Beta vulgaris spec. altissima, Beta vulgaris spec. rapa, Brassica napus var. napus, Brassica napus var. napobrassica, Brassica rapa var. silvestris, Brassica oleracea, Brassica nigra, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pistacia vera, Pisum sativum, Prunus avium, Prunus persica, Pyrus communis, Prunus armeniaca, Prunus cerasus, Prunus dulcis and Prunus domestica, Ribes sylvestre, Ricinus communis, Saccharum officinarum, Secale cereale, Sinapis alba, Solanum tuberosum, Sorghum bicolor (s. vulgare), Theobroma cacao, Trifolium pratense, Triticum aestivum, Triticale, Triticum durum, Vicia faba, Vitis vinifera and Zea mays.

Preferred crops are Arachis hypogaea, Beta vulgaris spec. altissima, Brassica napus var. napus, Brassica oleracea, Citrus limon, Citrus sinensis, Coffea arabica (Coffea canephora, Coffea liberica), Cynodon dactylon, Glycine max, Gossypium hirsutum, (Gossypium arboreum, Gossypium herbaceum, Gossypium vitifolium), Helianthus annuus, Hordeum vulgare, Juglans regia, Lens culinaris, Linum usitatissimum, Lycopersicon lycopersicum, Malus spec., Medicago sativa, Nicotiana tabacum (N. rustica), Olea europaea, Oryza sativa, Phaseolus lunatus, Phaseolus vulgaris, Pistacia vera, Pisum sativum, Prunus dulcis, Saccharum officinarum, Secale cereale, Solanum tuberosum, Sorghum bicolor (S. vulgare), Triticale, Triticum aestivum, Triticum durum, Vicia faba, Vitis vinifera and Zea mays.

Especially preferred crops are crops of cereals, corn, soybeans, rice, oilseed rape, cotton, potatoes, peanuts or permanent crops.

The form A of the compound of formula (I) according to the invention, or the agrochemical compositions comprising form A, can also be used in crops which have been modified by mutagenesis or genetic engineering in order to provide a new trait to a plant or to modify an already present trait.

Mutagenesis includes techniques of random mutagenesis using X-rays or mutagenic chemicals, but also techniques of targeted mutagenesis, in order to create mutations at a specific locus of a plant genome. Targeted mutagenesis techniques frequently use oligonucleotides or proteins like CRISPR/Cas, zinc-finger nucleases, TALENs or meganucleases to achieve the targeting effect.

Genetic engineering usually uses recombinant DNA techniques to create modifications in a plant genome which under natural circumstances cannot readily be obtained by cross breeding, mutagenesis or natural recombination. Typically, one or more genes are integrated into the genome of a plant in order to add a trait or improve a trait. These integrated genes are also referred to as transgenes in the art, while plant comprising such transgenes are referred to as transgenic plants. The process of plant transformation usually produces several transformation events, which differ in the genomic locus in which a transgene has been integrated. Plants comprising a specific transgene on a specific genomic locus are usually described as comprising a specific “event”, which is referred to by a specific event name. Traits which have been introduced in plants or have been modified include in particular herbicide tolerance, insect resistance, increased yield and tolerance to abiotic conditions, like drought.

Herbicide tolerance has been created by using mutagenesis as well as using genetic engineering. Plants which have been rendered tolerant to acetolactate synthase (ALS) inhibitor herbicides by conventional methods of mutagenesis and breeding comprise plant varieties commercially available under the name Clearfield®. However, most of the herbicide tolerance traits have been created via the use of transgenes.

Herbicide tolerance has been created to glyphosate, glufosinate, 2,4-D, dicamba, oxynil herbicides, like bromoxynil and ioxynil, sulfonylurea herbicides, ALS inhibitor herbicides and 4-hydroxyphenylpyruvate dioxygenase (HPPD) inhibitors, like isoxaflutole and mesotrione.

Transgenes which have been used to provide herbicide tolerance traits comprise: for tolerance to glyphosate: cp4 epsps, epsps grg23ace5, mepsps, 2mepsps, gat4601, gat4621 and goxv247, for tolerance to glufosinate: pat and bar, for tolerance to 2,4-D: aad-1 and aad-12, for tolerance to dicamba: dmo, for tolerance to oxynil herbicies: bxn, for tolerance to sulfonylurea herbicides: zm-hra, csr1-2, gm-hra, S4-HrA, for tolerance to ALS inhibitor herbicides: csr1-2, for tolerance to HPPD inhibitor herbicides: hppdPF, W336 and avhppd-03.

Transgenic corn events comprising herbicide tolerance genes are for example, but not excluding others, DAS40278, MON801, MON802, MON809, MON810, MON832, MON87411, MON87419, MON87427, MON88017, MON89034, NK603, GA21, MZHG0JG, HCEM485, VCO-Ø1981-5, 676, 678, 680, 33121, 4114, 59122, 98140, Bt10, Bt176, CBH-351, DBT418, DLL25, MS3, MS6, MZIR098, T25, TC1507 and TC6275.

Transgenic soybean events comprising herbicide tolerance genes are for example, but not excluding others, GTS 40-3-2, MON87705, MON87708, MON87712, MON87769, MON89788, A2704-12, A2704-21, A5547-127, A5547-35, DP356043, DAS44406-6, DAS68416-4, DAS-81419-2, GU262, SYHTØH2, W62, W98, FG72 and CV127.

Transgenic cotton events comprising herbicide tolerance genes are for example, but not excluding others, 19-51a, 31707, 42317, 81910, 281-24-236, 3006-210-23, BXN10211, BXN10215, BXN10222, BXN10224, MON1445, MON1698, MON88701, MON88913, GHB119, GHB614, LLCotton25, T303-3 and T304-40.

Transgenic canola events comprising herbicide tolerance genes are for example, but not excluding others, MON88302, HCR-1, HCN10, HCN28, HCN92, MS1, MS8, PHY14, PHY23, PHY35, PHY36, RF1, RF2 and RF3.

Insect resistance has mainly been created by transferring bacterial genes for insecticidal proteins to plants. Transgenes which have most frequently been used are toxin genes of Bacillus spec. and synthetic variants thereof, like cry1A, cry1Ab, cry1Ab-Ac, cry1Ac, cry1A.105, cry1F, cry1Fa2, cry2Ab2, cry2Ae, mcry3A, ecry3.1Ab, cry3Bb1, cry34Ab1, cry35Ab1, cry9C, vip3A(a), vip3Aa20. However, also genes of plant origin have been transferred to other plants. In particular genes coding for protease inhibitors, like CpTI and pinII. A further approach uses transgenes in order to produce double stranded RNA in plants to target and downregulate insect genes. An example for such a transgene is dvsnf7.

Transgenic corn events comprising genes for insecticidal proteins or double stranded RNA are for example, but not excluding others, Bt10, Bt11, Bt176, MON801, MON802, MON809, MON810, MON863, MON87411, MON88017, MON89034, 33121, 4114, 5307, 59122, TC1507, TC6275, CBH-351, MIR162, DBT418 and MZIR098.

Transgenic soybean events comprising genes for insecticidal proteins are for example, but not excluding others, MON87701, MON87751 and DAS-81419.

Transgenic cotton events comprising genes for insecticidal proteins are for example, but not excluding others, SGK321, MON531, MON757, MON1076, MON15985, 31707, 31803, 31807, 31808, 42317, BNLA-601, Event1, COT67B, COT102, T303-3, T304-40, GFM Cry1A, GK12, MLS 9124, 281-24-236, 3006-210-23, GHB119 and SGK321.

Increased yield has been created by increasing ear biomass using the transgene athb17, being present in corn event MON87403, or by enhancing photosynthesis using the transgene bbx32, being present in the soybean event MON87712.

Crops comprising a modified oil content have been created by using the transgenes: gm-fad2-1, Pj.D6D, Nc.Fad3, fad2-1A and fatb1-A. Soybean events comprising at least one of these genes are: 260-05, MON87705 and MON87769.

Tolerance to abiotic conditions, in particular to tolerance to drought, has been created by using the transgene cspB, comprised by the corn event MON87460 and by using the transgene Hahb-4, comprised by soybean event IND-00410-5.

Traits are frequently combined by combining genes in a transformation event or by combining different events during the breeding process. Preferred combination of traits are herbicide tolerance to different groups of herbicides, insect tolerance to different kind of insects, in particular tolerance to lepidopteran and coleopteran insects, herbicide tolerance with one or several types of insect resistance, herbicide tolerance with increased yield as well as a combination of herbicide tolerance and tolerance to abiotic conditions.

Plants comprising singular or stacked traits as well as the genes and events providing these traits are well known in the art. For example, detailed information as to the mutagenized or integrated genes and the respective events are available from websites of the organizations “International Service for the Acquisition of Agri-biotech Applications (ISAAA)” (http://www.isaaa.org/gmapprovaldatabase) and the “Center for Environmental Risk Assessment (CERA)” (http://cera-qmc.org/GMCropDatabase), as well as in patent applications, like EP3028573 and WO2017/011288.

The use of the compounds of formula (I) or formulations or combinations comprising them according to the invention on crops may result in effects which are specific to a crop comprising a certain gene or event. These effects might involve changes in growth behavior or changed resistance to biotic or abiotic stress factors. Such effects may in particular comprise enhanced yield, enhanced resistance or tolerance to insects, nematodes, fungal, bacterial, mycoplasma, viral or viroid pathogens as well as early vigour, early or delayed ripening, cold or heat tolerance as well as changed amino acid or fatty acid spectrum or content.

Furthermore, plants are also covered that contain by the use of recombinant DNA techniques a modified amount of ingredients or new ingredients, specifically to improve raw material production, e.g., potatoes that produce increased amounts of amylopectin (e.g. Amflora® potato, BASF SE, Germany).

Furthermore, it has been found that form A of formula (I) according to the invention, or the formulations comprising form A, are also suitable for the defoliation and/or desiccation of plant parts of crops such as cotton, potato, oilseed rape, sunflower, soybean or field beans, in particular cotton.

As desiccants, form A of formula (I) is particularly suitable for desiccating the above-ground parts of crop plants such as potato, oilseed rape, sunflower and soybean, but also cereals. This makes possible the fully mechanical harvesting of these important crop plants.

Also of economic interest is to facilitate harvesting, which is made possible by concentrating within a certain period of time the dehiscence, or reduction of adhesion to the tree, in citrus fruit, olives and other species and varieties of pernicious fruit, stone fruit and nuts. The same mechanism, i.e. the promotion of the development of abscission tissue between fruit part or leaf part and shoot part of the plants is also essential for the controlled defoliation of useful plants, in particular cotton.

Moreover, a shortening of the time interval in which the individual cotton plants mature leads to an increased fiber quality after harvesting.

Form A of the compound of formula (I) or the plant protection agents containing it can for example be used in the form of directly sprayable aqueous solutions, powders, suspensions and also high concentration aqueous, oily or other suspensions, oil suspensions, pastes, dusting agents, scattering agents or granules by spraying, misting, dusting, scattering or pouring. The use forms are determined by the use purposes; in each case, they should ensure the finest possible distribution of the active substances according to the invention.

The plant protection agents according to the invention contain compound of formula (I) in form A, i.e. in a purity, based on the modification in question, of at least 90 wt. %, and additives and/or carriers such as are usual for the formulation of plant protection agents. In such plant protection agents, the quantity of active substance, i.e. the total quantity of compound of formula (I) and of other active substances if necessary, normally lies in the range from 1 to 98 wt. %, in particular in the range from 5 to 95 wt. %, based on the total weight of the plant protection agent.

All solid and liquid substances which are normally used as carriers in plant protection agents, in particular in herbicide formulations are possible as carriers.

A formulation comprises a pesticidal effective amount of form A of the compound of formula (I).

The term “effective amount” denotes an amount of form A of the compound of formula (I), which is sufficient for controlling undesired vegetation, especially for controlling undesired vegetation in crops (i.e. cultivated plants) and which does not result in a substantial damage to the treated crop plants. Such an amount can vary in a broad range and is dependent on various factors, such as the undesired vegetation to be controlled, the treated crop plants or material, the climatic conditions and the specific form A of the compound of formula (I) used.

Form A of the compound of formula (I) can be converted into customary types of formulations, e. g. solutions, emulsions, suspensions, dusts, powders, pastes, granules, pressings, capsules, and mixtures thereof. Examples for formulation types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further formulation types are defined in the “Catalogue of pesticide formulation types and international coding system”, Technical Monograph No. 2, 6^{th} Ed. May 2008, CropLife International.

The formulations are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetting agents, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e. g. toluene, paraffin, tetrahydronaphthalene, alkylated naphthalenes; alcohols, e.g. ethanol, propanol, butanol, benzylalcohol, cyclohexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharides, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylarylsulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkylnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinylpyrrolidone, vinylalcohols, or vinylacetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide.

Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidal activity themselves, and which improve the biological performance of form A of the compound of formula (I) on the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e.g. xanthan gum, carboxymethylcellulose), inorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea, glycerin and salts like magnesium sulfate.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are polyvinylpyrrolidons, polyvinylacetates, polyvinyl alcohols, polyacrylates, biological or synthetic waxes, and cellulose ethers.

If the plant protection agents containing the crystalline modification A are used for seed treatment, they can also contain normal components such as are used for seed treatment, for example in dressing or coating. In addition to the aforesaid components, these include in particular colorants, adhesives, fillers and plasticizers.

All the dyes and pigments usual for such purposes are possible as colorants. Both pigments of low solubility in water and also dyes soluble in water are usable here. As examples, the dyes and pigments known under the names Rhodamin B, C.I. Pigment Red 112 and C.I. Solvent Red 1, Pigment Blue 15:4, Pigment Blue 15:3, Pigment Blue 15:2, Pigment Blue 15:1, Pigment Blue 80, Pigment Yellow 1, Pigment Yellow 13, Pigment Red 48:2, Pigment Red 48:1, Pigment Red 57:1, Pigment Red 53:1, Pigment Orange 43, Pigment Orange 34, Pigment Orange 5, Pigment Green 36, Pigment Green 7, Pigment White 6, Pigment Brown 25, Basic Violet 10, Basic Violet 49, Acid Red 51, Acid Red 52, Acid Red 14, Acid Blue 9, Acid Yellow 23, Basic Red 10, Basic Red 10 and Basic Red 108 may be mentioned. The quantity of colorant will normally not constitute more than 20 wt. % of the formulation and preferably lies in the range from 0.1 to 15 wt. %, based on the total weight of the formulation.

All binders normally usable in dressings come under consideration as adhesives. Examples of suitable binders include thermoplastic polymers such as poly-vinylpyrrolidone, polyvinyl acetate, polyvinyl alcohol and tylose and also polyacrylates, polymethacrylates, polybutenes, polyisobutenes, polystyrene, polyethylene amines, polyethylene amides, the aforesaid protective colloids, polyesters, polyether esters, polyanhydrides, polyester urethanes, polyester amides, thermoplastic polysaccharides, for example cellulose derivatives such as cellulose esters, cellulose ethers, cellulose ether esters, including methylcellulose, ethylcellulose, hydroxymethylcellulose, carboxymethylcellulose, hydroxypropyl cellulose and starch derivatives and modified starches, dextrins, maltodextrins, alginates and chitosans, and also fats, oils, proteins, including casein, gelatin and zein, gum Arabic and shellac. The adhesives are preferably plant-compatible, i.e. they exhibit no, or no significant, phytotoxic effects. The adhesives are preferably biodegradable. The adhesive is preferably selected such that it acts as a matrix for the active components of the formulation. The quantity of adhesive will normally not constitute more than 40 wt. % of the formulation and preferably lies in the range from 1 to 40 wt. % and in particular in the range from 5 to 30 wt. %, based on the total weight of the formulation.

In addition to the adhesive, the formulation for seed treatment can also contain inert fillers. Examples of these are the aforesaid solid carriers, in particular finely divided inorganic materials such as clays, chalk, bentonite, kaolin, talc, perlite, mica, silica gel, diatomaceous earth, quartz powder and montmorillonite but also fine-particle organic materials such as wood flour, cereal flour, active charcoal and the like. The quantity of filler is preferably selected such that the total quantity of filler does not exceed 70 wt. %, based on the total weight of all non-volatile components of the formulation. Often, the quantity of filler lies in the range from 1 to 50 wt. %, based on the total weight of all non-volatile components of the formulation.

In addition, the formulation for seed treatment can also contain a plasticizer which increases the flexibility of the coating. Examples of plasticizers are oligomeric polyalkylene glycols, glycerine, dialkyl phthalates, alkylbenzyl phthalates, glycol benzoates and comparable compounds. The quantity of plasticizer in the coating often lies in the range from 0.1 to 20 wt. %, based on the total weight of all non-volatile components of the formulation.

A preferred embodiment of the invention relates to liquid formulations of the form A. In addition to the solid active substance phase, these have at least one liquid phase, in which compound of formula (I) is present in form A in the form of dispersed fine particles.

Possible liquid phases are essentially water and those organic solvents in which form A is only slightly soluble, or insoluble, for example those wherein the solubility of form A at 25° C. and 1013 mbar is not more than 1 wt. %, in particular not more than 0.1 wt. %, and especially not more than 0.01 wt. %.

According to a first preferred embodiment, the liquid phase is selected from water and aqueous solvents, i.e. solvent mixtures which in addition to water also contain up to 20 wt. %, preferably however not more than 10 wt. %, based on the total quantity of water and solvent, of one or more organic solvents miscible with water, for example ethers miscible with water such as tetrahydrofuran, methyl glycol, methyl diglycol, alkanols such as isopropanol or polyols such as glycol, glycerine, diethylene glycol, propylene glycol and the like. Such formulations are also referred to below as suspension concentrates (SCs).

Such suspension concentrates contain compound of formula (I) as form A in a finely divided particulate form, wherein the particles of form A are present suspended in an aqueous phase. The size of the active substance particles, i.e. the size which 90 wt. % of the active substance particles do not exceed, here typically lies below 30 m, in particular below 20 m. Advantageously, in the SCs according to the invention, at least 40 wt. % and in particular at least 60 wt. % of the particles have diameters below 2 m.

In such SCs the quantity of active substance, i.e. the total quantity of compound of formula (I) and of other active substances if necessary, usually lies in the range from 10 to 70 wt. %, in particular in the range from 20 to 50 wt. %, based on the total weight of the suspension concentrate.

In addition to the active substance, aqueous suspension concentrates typically contain surface-active substances, and also if necessary antifoaming agents, thickeners (=rheology modifiers), antifreeze agents, stabilizers (biocides), agents for adjusting the pH and anticaking agents.

Possible surface-active substances are the previously named surface-active substances. Preferably the aqueous plant protection agents according to the invention contain at least one of the previously named anionic surfactants and if necessary one or more nonionic surfactants, if necessary in combination with a protective colloid. The quantity of surface-active substances will as a rule be 1 to 50 wt. %, in particular 2 to 30 wt. %, based on the total weight of the aqueous SCs according to the invention. Preferably the surface-active substances include at least one anionic surface-active substance and at least one nonionic surface-active substance, and the proportion of anionic to nonionic surface-active substance typically lies in the range from 10:1 to 1:10.

Concerning the nature and quantity of the antifoaming agents, thickeners, antifreeze agents and biocides, the same applies as aforesaid.

If necessary, the aqueous SCs according to the invention can contain buffers for pH regulation. Examples of buffers are alkali metal salts of weak inorganic or organic acids, such as for example phosphoric acid, boric acid, acetic acid, propionic acid, citric acid, fumaric acid, tartaric acid, oxalic acid and succinic acid.

According to a second preferred embodiment, the liquid phase consists of non-aqueous organic solvents in which the solubility of the form A of compound of formula (I) at 25° C. and 1013 mbar is not more than 1 wt. %, in particular not more than 0.1 wt. %, and especially not more than 0.01 wt. %. These include in particular aliphatic and cycloaliphatic hydrocarbons and oils, in particular those of plant origin, and also C₁-C₄ alkyl esters of saturated or unsaturated fatty acids or fatty acid mixtures, in particular the methyl esters, for example methyl oleate, methyl stearate and rape oil methyl ester, but also paraffinic mineral oils and the like.

Accordingly, the present invention relates also to agents for plant protection in the form of a non-aqueous suspension concentrate, which will also be referred to below as OD (oil-dispersion). Such ODs contain form A of compound of formula (I) in a finely divided particulate form, wherein the particles of form A are present suspended in a non-aqueous phase. The size of the active substance particles, i.e. the size which 90 wt. % of the active substance particles do not exceed, here typically lies below 30 μm, in particular below 20 μm.

Advantageously, in the non-aqueous suspension concentrates, at least 40 wt. % and in particular at least 60 wt. % of the particles have diameters below 2 m.

In such ODs, the quantity of active substance, i.e. the total quantity of compound of formula (I) and of other active substances if necessary, usually lies in the range from 10 to 70 wt. %, in particular in the range from 20 to 50 wt. %, based on the total weight of the non-aqueous suspension concentrate.

In addition to the active substance and the liquid carrier, non-aqueous suspension concentrates typically contain surface-active substances, and also if necessary antifoaming agents, agents to modify the rheology and stabilizers (biocides).

Possible surface-active substances are preferably the previously named anionic and nonionic surfactants. The quantity of surface-active substances will as a rule be 1 to 30 wt. %, in particular 2 to 20 wt. %, based on the total weight of the non-aqueous SCs according to the invention. Preferably the surface-active substances include at least one anionic surface-active substance and at least one nonionic surface-active substance, and the proportion of anionic to nonionic surface-active substance typically lies in the range from 10:1 to 1:10.

Form A of the compound of formula (I) according to the invention can also be formulated as solid plant protection agents. These include powder, scattering and dusting agents but also water-dispersible powders and granules, for example coated, impregnated and homogenous granules.

Such formulations can be produced by mixing or simultaneous grinding of form A of the compound of formula (I) with a solid carrier and if necessary other additives, in particular surface-active substances. Granules can be produced by binding of the active substances to solid carriers. Solid carriers are mineral earths such as silicic acids, silica gels, silicates, talc, kaolin, limestone, lime, chalk, bole, loess, clay, dolomite, diatomaceous earth, calcium and magnesium sulfate, magnesium oxide, ground plastics, fertilizers such as ammonium sulfate, ammonium phosphate, ammonium nitrate, ureas and plant products such as cereal flour, tree bark, wood and nutshell flour, cellulose powder or other solid carriers. Solid formulations can also be produced by spray drying, if necessary in the presence of polymeric or inorganic drying aids, and if necessary in the presence of solid carriers.

For the production of solid formulations of the compound of formula (I) in form A, extrusion processes, fluidized bed granulation, spray granulation and comparable technologies are suitable.

Possible surface-active substances are the previously named surfactants and protective colloids. The quantity of surface-active substances will as a rule be 1 to 30 wt. %, in particular 2 to 20 wt. %, based on the total weight of the solid formulation according to the invention.

In such solid formulations, the quantity of active substance, i.e. the total quantity of compound of formula (I) and of other active substances if necessary, usually lies in the range from 10 to 70 wt. %, in particular in the range from 20 to 50 wt. %, based on the total weight of the solid formulation.

Examples for agrochemical composition types and their preparation are

### i) Water-Soluble Concentrates (SL, LS)

10-60 wt % of form A of compound of formula (I) according to the invention and 5-15 wt % wetting agent (e.g. alcohol alkoxylates) are dissolved in water and/or in a water-soluble solvent (e.g. alcohols) ad 100 wt %. The active substance dissolves upon dilution with water.

### ii) Dispersible Concentrates (DC)

5-25 wt % of form A of compound of formula (I) according to the invention and 1-10 wt % dispersant (e. g. polyvinylpyrrolidone) are dissolved in organic solvent (e.g. cyclohexanone) ad 100 wt %. Dilution with water gives a dispersion.

iii) Suspensions (SC, OD, FS)

In an agitated ball mill, 20-60 wt % of form A of compound of formula (I) according to the invention are comminuted with addition of 2-10 wt % dispersants and wetting agents (e.g. sodium lignosulfonate and alcohol ethoxylate), 0, 1-2 wt % thickener (e.g. xanthan gum) and water ad 100 wt % to give a fine active substance suspension. Dilution with water gives a stable suspension of the active substance. For FS type formulation up to 40 wt % binder (e.g. polyvinylalcohol) is added.

### iv) Water-Dispersible Granules and Water-Soluble Granules (WG, SG)

50-80 wt % of form A of compound of formula (I) according to the invention are ground finely with addition of dispersants and wetting agents (e.g. sodium lignosulfonate and alcohol ethoxylate) ad 100 wt % and prepared as water-dispersible or water-soluble granules by means of technical appliances (e. g. extrusion, spray tower, fluidized bed). Dilution with water gives a stable dispersion or solution of the active substance.

### v) Water-Dispersible Powders and Water-Soluble Powders (WP, SP, WS)

50-80 wt % of form A of compound of formula (I) according to the invention are ground in a rotor-stator mill with addition of 1-5 wt % dispersants (e.g. sodium lignosulfonate), 1-3 wt % wetting agents (e.g. alcohol ethoxylate) and solid carrier (e.g. silica gel) ad 100 wt %. Dilution with water gives a stable dispersion or solution of the active substance.

### vi) Gel (GW, GF)

In an agitated ball mill, 5-25 wt % of form A of compound of formula (I) according to the invention are comminuted with addition of 3-10 wt % dispersants (e.g. sodium lignosulfonate), 1-5 wt % thickener (e.g. carboxymethylcellulose) and water ad 100 wt % to give a fine suspension of the active substance. Dilution with water gives a stable suspension of the active substance.

vii) Microcapsules (CS)

An oil phase comprising 5-50 wt % of form A of compound of formula (I) according to the invention, 0-40 wt % water insoluble organic solvent (e.g. aromatic hydrocarbon), 2-15 wt % acrylic monomers (e.g. methylmethacrylate, methacrylic acid and a di- or triacrylate) are dispersed into an aqueous solution of a protective colloid (e.g. polyvinyl alcohol). Radical polymerization initiated by a radical initiator results in the formation of poly(meth)acrylate microcapsules. Alternatively, an oil phase comprising 5-50 wt % of form A of compound of formula (I), 0-40 wt % water insoluble organic solvent (e.g. aromatic hydrocarbon), and an isocyanate monomer (e.g. diphenylmethene-4,4′-diisocyanate) are dispersed into an aqueous solution of a protective colloid (e.g. polyvinyl alcohol). The addition of a polyamine (e.g. hexamethylenediamine) results in the formation of polyurea microcapsules. The monomers amount to 1-10 wt %. The wt % relate to the total CS formulation.

viii) Dustable Powders (DP, DS)

1-10 wt % of form A of compound of formula (I) according to the invention are ground finely and mixed intimately with solid carrier (e.g. finely divided kaolin) ad 100 wt %.

### ix) Granules (GR, FG)

0.5-30 wt % of form A of compound of formula (I) according to the invention is ground finely and associated with solid carrier (e.g. silicate) ad 100 wt %. Granulation is achieved by extrusion, spray-drying or the fluidized bed.

The formulation types i) to ix) may optionally comprise further auxiliaries, such as 0, 1-1 wt % bactericides, 5-15 wt % anti-freezing agents, 0, 1-1 wt % anti-foaming agents, and 0, 1-1 wt % colorants.

The application of the form A or the herbicidal agents containing it is effected, if the formulation is not already ready for use, in the form of aqueous spray fluids. These are prepared by dilution of the aforesaid formulations containing the form A with water. The spray fluids can also contain other components in dissolved, emulsified or suspended form, for example fertilizers, active substances of other herbicidal or growth-regulating active substance groups, other active substances, for example active substances for combating animal pests or phyto-pathogenic fungi or bacteria, and also mineral salts which are used for the elimination of nutritional and trace element deficiencies, and non-phytotoxic oils and oil concentrates. As a rule, these components are added to the spray fluid before, during or after the dilution of the formulations according to the invention.

The application of the form A or of the plant protection agents containing them can be effected in a pre-emergence or in a post-emergence method. If compound of formula (I) is less tolerable for certain crop plants, application techniques can be used wherein the herbicidal agents are sprayed using the spraying equipment in such a manner that the leaves of the sensitive crop plants are as far as possible not hit, while the active substances reach the leaves of undesired plants growing under them or the uncovered soil surface (post-directed, lay-by).

The quantities of compound of formula (I) applied are 0.001 to 3.0 kg active substance per hectare, preferably 0.005 to 0.5 kg active substance (a·S)/ha, depending on the treatment aim, season, target plants and growth stage.

In a further embodiment, the application of the form A or the plant protection agent containing them can be effected by treatment of seed.

Treatment of seed essentially includes all techniques with which the person skilled in the art is familiar (seed dressing, seed coating, seed dusting, seed soaking, seed film coating, seed multilayer coating, seed encrusting, seed dripping and seed pelleting) on the basis of compound of formula (I) in form A or B, or agents prepared therefrom. Here the plant protection agents can be applied diluted or undiluted.

The term seed includes seed of all types, for example grains, seeds, fruits, tubers, cuttings and similar forms. Preferably, the term seed here describes grains and seeds.

As seed, seed of the crop plants mentioned above but also the seeds of transgenic plants or those obtained by conventional breeding methods can be used.

For the seed treatment, form A of the compound of formula (I) is normally used in quantities of 0.001 to 10 kg per 100 kg of seed.

### A PREPARATION EXAMPLES

### Prep. Example 1

### Methyl 2-[2-[2-bromo-4-fluoro-5-[3-methyl-2,6-dioxo-4-(trifluoromethyl)pyrimidin-1-yl]phenoxy]phenoxy]-2-methoxy-acetate

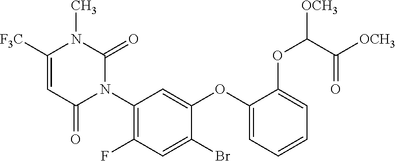 [SMILES: COC(=O)C(OC)Oc1ccccc1Oc1cc(-n2c(=O)cc(C(F)(F)F)n(C)c2=O)c(F)cc1Br]

### Prep. Example 1—Step 1: tert-Butyl N-(2,5-difluoro-4-nitro-phenyl)carbamate

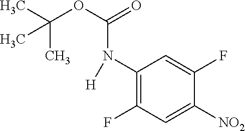 [SMILES: CC(C)(C)OC(=O)Nc1cc(F)c([N+](=O)[O-])cc1F]

4-Dimethylaminopyridine (12.2 g, 100 mmol) was added to a solution of 2,5-difluoro-4-nitro-aniline (CAS 1542-36-5; 172 g, 1 mol), bis-(1,1-dimethylethyl) dicarbonate (327 g, 1.5 mol) in dichloromethane (2 L) at 25° C. The mixture was stirred at 25° C. for 18 h. The resulting mixture was concentrated and purified with silica gel column (ethylacetate: petrolether 1:9) to give tert-butyl N-(2,5-difluoro-4-nitro-phenyl)carbamate (250 g, 91.2%) as yellow solid.

¹H NMR (CDCl₃ 400 MHz): δ ppm=8.27 (dd, J=13.1, 6.6 Hz, 1H), 7.91 (dd, J=10.6, 6.6 Hz, 1H), 7.05 (br s, 1H), 1.57 (s, 9H).

### Prep. Example 1—Step 2: tert-butyl N-[2-fluoro-5-(2-methoxyphenoxy)-4-nitro-phenyl]carbamate

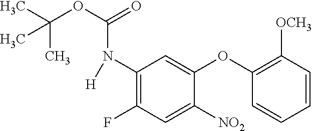 [SMILES: COc1ccccc1Oc1cc(NC(=O)OC(C)(C)C)c(F)cc1[N+](=O)[O-]]

To a solution of tert-butyl N-(2,5-difluoro-4-nitro-phenyl)carbamate (250 g, 911 mmol) and K₂CO₃ (377 g, 2733 mmol) in acetonitrile (2.5 L) was added 2-methoxyphenol (136 g, 1094 mmol) at 15° C. Then the mixture was stirred at 80° C. for 18 h. The mixture was filtered and the filtrate was concentrated. The residue was diluted with ethylacetate, washed with H₂O, brine, dried over Na₂SO₄ and concentrated. The residue was triturated with ethylacetate:petrolether 1:3 (1 L) to give tert-butyl N-[2-fluoro-5-(2-methoxyphenoxy)-4-nitro-phenyl]carbamate (220 g, 64%) as yellow solid.

¹H NMR (400 MHz, DMSO-d6) δ ppm=9.63 (s, 1H) 8.04 (d, J=10.6 Hz, 1H), 7.45 (d, J=6.7 Hz, 1H), 7.19-7.29 (m, 2H), 7.13 (d, J=7.7 Hz, 1H), 6.98-7.03 (m, 1H), 3.74 (s, 3H), 1.37 (s, 9H).

### Prep. Example 1—Step 3: tert-butyl N-[4-amino-2-fluoro-5-(2-methoxyphenoxy)phenyl]-carbamate

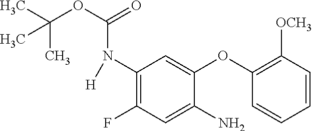 [SMILES: COc1ccccc1Oc1cc(NC(=O)OC(C)(C)C)c(F)cc1N]

To the solution of tert-butyl N-[2-fluoro-5-(2-methoxyphenoxy)-4-nitro-phenyl]carbamate (210 g, 555 mmol) in ethanol (3.6 L) was added Pd/C (21 g) under N₂ and stirred at 25° C. under H₂ (50 Psi) for 24 h. The mixture was filtered and concentrated to give tert-butyl N-[4-amino-2-fluoro-5-(2-methoxyphenoxy)phenyl]carbamate (170 g, 80.6%) as a brown solid.

¹H NMR (400 MHz, DMSO-d6) δ ppm=8.34 (br s, 1H), 7.06-7.15 (m, 2H), 6.86-6.93 (m, 1H), 6.78-6.84 (m, 1H), 6.61 (br s, 1H), 6.55 (d, J=12.1 Hz, 1H), 5.02 (s, 2H), 3.79 (s, 3H), 1.36 (s, 9H).

### Prep. Example 1—Step 4: tert-butyl N-[4-bromo-2-fluoro-5-(2-methoxyphenoxy)phenyl]carbamate

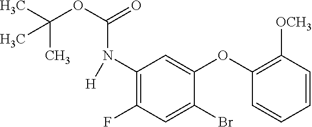 [SMILES: COc1ccccc1Oc1cc(NC(=O)OC(C)(C)C)c(F)cc1Br]

To the mixture of CuBr₂ (26.6 g, 120 mmol) in acetonitrile (200 mL) was added isoamyl nitrite (10.5 g, 90 mmol) at 25° C. Then, the mixture was warmed to 60° C. tert-butyl N-[4-amino-2-fluoro-5-(2-methoxyphenoxy)phenyl]-carbamate (31 g, 60 mmol) was added in portions at 60° C. and stirred for 1 h at 60° C. Then the mixture was diluted with H₂O, extracted with ethylacetate twice. The organic layer was washed with brine, dried over Na₂SO₄ and concentrated. The crude was purified by column (ethylacetate:petrolether 1:4) to give tert-butyl N-[4-bromo-2-fluoro-5-(2-methoxyphenoxy)phenyl]carbamate (13 g, 52.5%) as a brown solid.

¹H NMR (400 MHz, CDCl₃) δ ppm=7.72 (br s, 1H), 7.33 (d, J=10.2 Hz, 1H), 7.13-7.07 (m, 1H), 7.02-6.99 (m, 1H), 6.92-6.86 (m, 1H), 6.83-6.79 (m, 1H), 6.61 (br s, 1H), 3.88 (s, 3H), 1.45 (s, 9H).

### Prep. Example 1—Step 5: 4-bromo-2-fluoro-5-(2-methoxyphenoxy)aniline

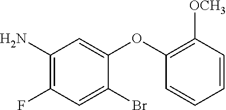 [SMILES: COc1ccccc1Oc1cc(N)c(F)cc1Br]

To tert-butyl N-[4-bromo-2-fluoro-5-(2-methoxyphenoxy)phenyl]carbamate (3 g, 7.3 mmol) was added 4N HCl in ethylacetate (30 mL) in portions at 0° C. and stirred for 16 h at 20° C. The mixture was poured into H₂O, extracted with ethylacetate, and the organic layer was washed with brine, dried over Na₂SO₄ and concentrated to give 4-bromo-2-fluoro-5-(2-methoxyphenoxy)-aniline (2.3 g, crude) as a brown solid.

¹H NMR (400 MHz, CDCl₃) δ ppm=7.23 (d, J=10.2 Hz, 1H), 7.15-7.09 (m, 1H), 7.01 (dd, J=1.2, 8.1 Hz, 1H), 6.93-6.88 (m, 1H), 6.87-6.83 (m, 1H), 6.27 (d, J=8.2 Hz, 1H), 3.87 (s, 3H), 3.69 (br s, 2H).

### Prep. Example 1—Step 6: 3-[4-bromo-2-fluoro-5-(2-methoxyphenoxy)phenyl]-6-(trifluoromethyl)-1H-pyrimidine-2,4-dione

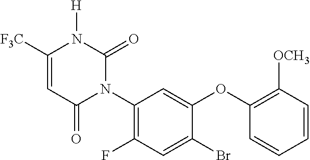 [SMILES: COc1ccccc1Oc1cc(-n2c(=O)cc(C(F)(F)F)[nH]c2=O)c(F)cc1Br]

To a solution of 4-bromo-2-fluoro-5-(2-methoxyphenoxy)aniline (1.8 g, 5.8 mol) in acetic acid (5 mL) was added 2-(dimethylamino)-4-(trifluoromethyl)-1,3-oxazin-6-one (CAS 141860-79-9, 1.8 g, 8.7 mmol) at 20° C. The mixture was stirred at 75° C. for 16 h. The mixture was poured into water and extracted with ethylacetate. The organic layer was washed with brine, dried over Na₂SO₄ and concentrated to give 3-[4-bromo-2-fluoro-5-(2-methoxyphenoxy)phenyl]-6-(trifluoromethyl)-1H-pyrimidine-2,4-dione (3.2 g, crude) as a black solid.

### Prep. Example 1—Step 7: 3-[4-bromo-2-fluoro-5-(2-methoxyphenoxy)phenyl]-1-methyl-6-(trifluoro-methyl)-pyrimidine-2,4-dione

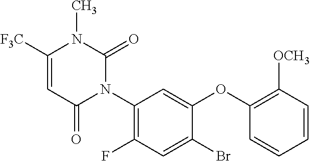 [SMILES: COc1ccccc1Oc1cc(-n2c(=O)cc(C(F)(F)F)n(C)c2=O)c(F)cc1Br]

To a mixture of 3-[4-bromo-2-fluoro-5-(2-methoxyphenoxy)phenyl]-6-(trifluoromethyl)-1H-pyrimidine-2,4-dione (4.1 g, 8.6 mmol) in acetonitrile (40 mL) was added K₂CO₃ (4.7 g, 34.2 mmol) and methyliodide (2.5 g, 17.3 mmol) dropwise with stirring at 25° C. Then, it was stirred for 16 h at 60° C. The mixture was filtered, and the filtrate was concentrated. The crude was triturated. with ethylacetate:petrolether 1:10 (30 mL) to give 3-[4-bromo-2-fluoro-5-(2-methoxyphenoxy)phenyl]-1-methyl-6-(trifluoromethyl)pyrimidine-2,4-dione (3.4 g, crude) as a yellow solid.

¹H NMR (400 MHz, CDCl₃) δ ppm=7.54 (d, J=8.7 Hz, 1H), 7.20-7.14 (m, 1H), 7.05 (dd, J=1.5, 7.9 Hz, 1H), 7.00 (dd, J=1.2, 8.2 Hz, 1H), 6.97-6.92 (m, 1H), 6.57 (d, J=6.4 Hz, 1H), 6.30 (s, 1H), 3.81 (s, 3H), 3.51 (s, 3H).

### Prep. Example 1 Step 8: 3-[4-bromo-2-fluoro-5-(2-hydroxyphenoxy)phenyl]-1-methyl-6-(trifluoro-methyl)-pyrimidine-2,4-dione

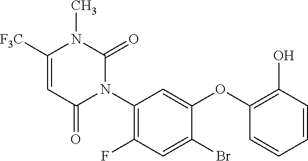 [SMILES: Cn1c(C(F)(F)F)cc(=O)n(-c2cc(Oc3ccccc3O)c(Br)cc2F)c1=O]

To a solution of 3-[4-bromo-2-fluoro-5-(2-methoxyphenoxy)phenyl]-1-methyl-6-(trifluoro-methyl)pyrimidine-2,4-dione (3.4 g, 6.9 mmol) in CH₂Cl₂ (50 mL) was added BBr₃ (3.5 g, 13.9 mmol) dropwise with stirring at 0° C. The mixture was stirred at 25° C. for 2 h. The mixture was poured into ice water and extracted with ethylacetate. The organic layer was washed with brine (100 mL), dried over Na₂SO₄, filtered and concentrated to give 3-[4-bromo-2-fluoro-5-(2-hydroxyphenoxy)phenyl]-1-methyl-6-(trifluoromethyl)pyrimidine-2,4-dione (2.3 g, 66% over steps 6, 7 and 8) as a yellow solid.

¹H NMR (400 MHz, CDCl₃-d) δ ppm=7.56 (d, J=8.8 Hz, 1H), 7.08-7.01 (m, 2H), 6.90-6.81 (m, 3H), 6.31 (s, 1H), 5.66 (br s, 1H), 3.53-3.50 (s, 3H).

### Prep. Example 1—Step 9: methyl 2-[2-[2-bromo-4-fluoro-5-[3-methyl-2,6-dioxo-4-(trifluoromethyl)-pyrimidin-1-yl]phenoxy]phenoxy]-2-methoxy-acetate

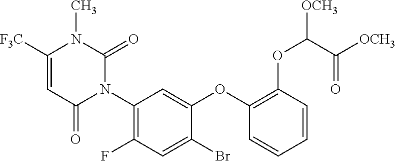 [SMILES: COC(=O)C(OC)Oc1ccccc1Oc1cc(-n2c(=O)cc(C(F)(F)F)n(C)c2=O)c(F)cc1Br]

To a solution of 3-[4-bromo-2-fluoro-5-(2-hydroxyphenoxy)phenyl]-1-methyl-6-(trifluoromethyl)-pyrimidine-2,4-dione (1 g, 2.1 mmol) in DMF (10 mL) was added Cs₂CO₃ (2.1 g, 6.3 mmol), methyl 2-bromo-2-methoxyacetate (CAS 5193-96-4, 772 mg, 4.2 mol) at 10° C. Then the reaction was stirred at 10° C. for 16 h. The mixture was poured into water and extracted with ethylacetate. The organic layer was washed with brine (30 ml), dried over anhydrous Na₂SO₄, concentrated. The crude was purified by column (ethylacetate:petrolether 1:5) and by prep-HPLC (acetonitrile—H₂O) to give methyl 2-[2-[2-bromo-4-fluoro-5-[3-methyl-2,6-dioxo-4-(trifluoromethyl)-pyrimidin-1-yl]phenoxy]phenoxy]-2-methoxy-acetate (0.285 g, 23%) as white solid.

¹H NMR (400 MHz, DMSO-d6) δ ppm=7.94 (d, J=8.8 Hz, 1H), 7.23 (d, J=1.8 Hz, 1H), 7.23-7.18 (m, 1H), 7.15-7.10 (m, 1H), 7.08-7.04 (m, 1H), 6.96 (d, J=6.6 Hz, 1H), 6.50 (d, J=1.8 Hz, 1H), 5.67 (d, J=1.8 Hz, 1H), 3.68 (d, J=1.8 Hz, 3H), 3.35 (s, 3H), 3.29 (d, J=6.6 Hz, 3H).

### B USE EXAMPLES

The herbicidal activity of form A of the compound of formula (I) was demonstrated by the following greenhouse experiments:

The culture containers used were plastic flowerpots containing loamy sand with approximately 3.0% of humus as the substrate. The seeds of the test plants were sown separately for each species.

For the pre-emergence treatment, the active ingredients, which had been suspended or emulsified in water, were applied directly after sowing by means of finely distributing nozzles.

The containers were irrigated gently to promote germination and growth and subsequently covered with transparent plastic hoods until the test plants had rooted. This cover caused uniform germination of the test plants, unless this had been impaired by the active ingredients.

For the post-emergence treatment, the test plants were first grown to a height of 3 to 15 cm, depending on the plant habit, and only then treated with the active ingredients which had been suspended or emulsified in water. For this purpose, the test plants were either sown directly and grown in the same containers, or they were first grown separately as seedlings and transplanted into the test containers a few days prior to treatment.

Depending on the species, the test plants were kept at 10-25° C. or 20-35° C., respectively. The test period extended over 2 to 4 weeks. During this time, the test plants were tended, and their response to the individual treatments was evaluated.

Evaluation was carried out using a scale from 0 to 100. 100 means no emergence of the test plants, or complete destruction of at least the aerial moieties, and 0 means no damage, or normal course of growth. A good herbicidal activity is given at values of at least 70 and a very good herbicidal activity is given at values of at least 85.

The abovementioned methods were used to compare, in a greenhouse test, form A according to the invention and, as comparison the compound of formula (I) in amorphous form, in each case formulated as aqueous suspension concentrate (SC; 8.33 w/v %).

The test plants used in the greenhouse experiments were of the following species:

| Bayer code | Scientific name |
|---|---|
| ERICA | Erigeron canadensis |
| LOLMU | Lolium multiflorum |

TABLE 2
Comparison of the herbicidal activity of form A with the
amorphous form [each of the compound of formula (I)]
applied post-emergence (greenhouse) and assessment 20 DAT

| | Application rate | Form A | amorphous |
|---|---|---|---|
| Test plants | (g/ha a.i.) | Damage [%] | |
| Erigeron canadensis | 4 | 100 | 70 |
| | 2 | 60 | 30 |
| Lolium multiflorum | 16 | 90 | 80 |
| | 8 | 90 | 75 |
| | 4 | 70 | 25 |
| | 2 | 50 | 15 |

1. A crystalline form A of methyl 2-[2-[2-bromo-4-fluoro-5-[3-methyl-2,6-dioxo-4-(trifluoromethyl)pyrimidin-1-yl]phenoxy]phenoxy]-2-methoxy-acetate, which in an X-ray powder diffraction diagram at 25° C. and Cu—Kα radiation displays at least 3 of the following reflections, quoted as 2θ values: 7.6±0.2, 8.9±0.2, 9.3±0.2, 11.2±0.2, 12.7±0.2, 13.4±0.2, 14.3±0.2, 15.8±0.2, 16.6±0.2, 17.6±0.2, 18.6±0.2, 19.1±0.2, 19.8±0.2, 20.4±0.2, 21.0±0.2, 21.4±0.2, 21.9±0.2, 22.5±0.2, 22.6±0.2, 23.0±0.2, 23.6±0.2, 24.7±0.2, 25.5±0.2, 26.3±0.2, 26.6±0.2, 27.1±0.2, 27.9±0.2, 28.5±0.2, 29.0±0.2, 29.4±0.2.

2. The crystalline form A as claimed in claim 1, which in an X-ray powder diffraction diagram at 25° C. and Cu—Kα radiation displays the following reflections, quoted as 2θ values 9.3±0.2, 11.2±0.2, 13.4±0.2, 16.6±0.2, 19.8±0.2, 21.9±0.2 and 22.5±0.2.

3. The crystalline form A as claimed in claim 1 with a content of methyl 2-[2-[2-bromo-4-fluoro-5-[3-methyl-2,6-dioxo-4-(trifluoromethyl)pyrimidin-1-yl]phenoxy]phenoxy]-2-methoxy-acetate of at least 94 wt. %.

4. Methyl 2-[2-[2-bromo-4-fluoro-5-[3-methyl-2,6-dioxo-4-(trifluoromethyl)pyrimidin-1-yl]phenoxy]phenoxy]-2-methoxy-acetate consisting of at least 90 wt. % of the crystalline form A as claimed in claim 1.

5. A process to produce the crystalline form A as claimed in claim 1, comprising:
i) preparation of a solution of methyl 2-[2-[2-bromo-4-fluoro-5-[3-methyl-2,6-dioxo-4-(trifluoromethyl)pyrimidin-1-yl]phenoxy]phenoxy]-2-methoxy-acetate,
ii) effecting a crystallization of methyl 2-[2-[2-bromo-4-fluoro-5-[3-methyl-2,6-dioxo-4-(trifluoromethyl)pyrimidin-1-yl]phenoxy]phenoxy]-2-methoxy-acetate.

6. A plant protection agent containing methyl 2-[2-[2-bromo-4-fluoro-5-[3-methyl-2,6-dioxo-4-(trifluoromethyl)pyrimidin-1-yl]phenoxy]phenoxy]-2-methoxy-acetate which consists of at least 90 wt. % of the crystalline form A as claimed in claim 1, and one or more additives customary for the formulation of plant protection agents.

7. The plant protection agent as claimed in claim 6 in the form of an aqueous suspension concentrate.

8. The plant protection agent as claimed in claim 6 in the form of a non-aqueous suspension concentrate.

9. The plant protection agent as claimed in claim 6 in the form of a powder or granules dispersible in water.

10. A method for combating undesired plant growth, wherein methyl 2-[2-[2-bromo-4-fluoro-5-[3-methyl-2,6-dioxo-4-(trifluoromethyl)pyrimidin-1-yl]phenoxy]phenoxy]-2-methoxy-acetate consisting of at least 90 wt. % of the crystalline form A as claimed in claim 1 is used on plants, the habitat thereof, and/or on seeds.
